(19) United States
(12) Patent Application Publication
(10) Pub. No.: US 2026/0108732 A1
(43) Pub. Date: Apr. 23, 2026

(54) MASK-TYPE COSMETIC DEVICE

(71) Applicant: YA-MAN LTD., Tokyo (JP)

(72) Inventors: Kazunori YAMANAKA, Tokyo (JP); Masatake KISHI, Tokyo (JP)

(21) Appl. No.: 19/341,429

(22) Filed: Sep. 26, 2025

(30) Foreign Application Priority Data
Oct. 17, 2024 (JP) 2024-182127
Feb. 14, 2025 (JP) 2025-022018
May 23, 2025 (JP) 2025-086198

(51) Int. Cl. A61N 1/32; A61N 1/04; A61N 1/36; A61N 5/06

(52) U.S. Cl. CPC A61N 1/328; A61N 1/0472; A61N 1/36014; A61N 5/0616; A61N 2005/0634; A61N 2005/0647; A61N 2005/0652

(57) ABSTRACT

[Problem] To provide a mask-type cosmetic device which, when removed from a face, can be folded and stored even in a small space and which prevents dust, etc., from adhering to inner sections thereof.

[Solution] A mask-type cosmetic device which can be worn on the face, wherein: the mask-type cosmetic device includes a housing which covers an exterior section, stimulation sections including electrical stimulation sections which apply electrical stimulation to the face and/or light-emitting sections which irradiate light onto the face, a controller which controls the stimulation sections, and a fixing means which is provided to the housing and fixes the mask-type cosmetic device to the face; a section that is worn on the face and includes the housing is configured so as to be foldable; and the housing has provided thereto a storage section for storing the controller integrally with the housing or separately therefrom in a detachable manner.

### TECHNICAL FIELD

The present disclosure relates to a mask-type cosmetic device.

### BACKGROUND

As disclosed in Patent Document 1 listed below, conventionally-known skin treatment apparatuses include apparatuses in which a cosmetic mask that can be worn on the face is provided with stimulators that apply electrical or ultrasonic stimulation.

Furthermore, Patent Document 1 discloses providing: a cosmetic mask which is easily attached/detached and enhances cosmetic effects; and an electric stimulator which can be applied to the cosmetic mask.

### CITATION LIST

### Patent Literature

Patent Document 1: JP 2022-042422 A

### SUMMARY OF INVENTION

### Technical Problem

However, the feature in Patent Document 1 described above had an issue in that when the cosmetic mask is removed from the face and stored, etc., since the body thereof is bulky, storage in a small space is difficult. Furthermore, the feature in Patent Document 1 also had a problem in that when the mask is left, dust, etc., adheres to inner sections thereof.

Thus, the objective of the feature according to the present disclosure is to obtain a mask-type cosmetic device which, when the mask body is removed, can be folded and stored even in a small space and also in which dust, etc., does not adhere to inner sections thereof.

### Solution to Problem

(1) One aspect is a mask-type cosmetic device that can be worn on a face, wherein: the mask-type cosmetic device includes a housing which covers an exterior section, stimulation sections including electrical stimulation sections which apply electrical stimulation to the face and/or light-emitting sections which irradiate light onto the face, a controller which controls the stimulation sections, and a fixing means which is provided to the housing and fixes the mask-type cosmetic device to the face; and a section that is worn on the face and includes the housing is configured so as to be foldable.
(2) In (1) above, the housing has provided thereto a storage section for storing the controller integrally with the housing or separately therefrom in a detachable manner.
(3) In (1) above, the fixing means is configured as left and right fixing belts which are integral with left and right sections of the housing or are provided in a detachable manner thereto, and one of the fixing belts has provided thereto a storage section for storing the controller integrally with the fixing belt or separately therefrom in a detachable manner.
(4) In (1) above, the housing has foldable folding sections.
(5) In (4) above, an inner angle of at least some of the folding sections is from 0 degrees to no more than 45 degrees.
(6) In (4) above, the housing is formed from a flexible material and has a configuration in which a plurality of substrates are disposed, on an inner surface of the housing, adjacent to one another and separated by fine grooves, and the grooves between the plurality of substrates constitute the folding sections.
(7) In (4) above, the housing has a configuration in which a plurality of polygonal regions are provided adjacent to one another and fine grooves are formed between each of the regions, and sections where the fine grooves are formed are rendered thin so as to constitute the folding sections.
(8) In (4) above, the housing has a configuration in which a plurality of polygonal regions are provided adjacent to one another and fine grooves are formed between each of the regions, and sections where the fine grooves are formed have a stiffness that is lower than the stiffness of the regions so as to constitute the folding sections.
(9) In (4) above, the mask-type cosmetic device is folded flat when not used and is opened out to a three-dimensional shape when used.
(10) In (9) above, the folding sections are disposed such that, when used, the mask-type cosmetic device is opened out to a three-dimensional shape by pulling out the housing.
(11) In (9) above, the mask-type cosmetic device is configured such that size thereof when the housing is folded flat is no more than 50% the size thereof when the housing is opened out to a three-dimensional shape.
(12) In (1) above, a flexible substrate is disposed on an inner surface-side of the housing, and a plurality of LEDs which constitute the light-emitting sections are disposed on the flexible substrate.
(13) In (12) above, a transmissive cover is disposed on an inner surface-side of the flexible substrate, and electrodes constituting some of the electrical stimulation sections are disposed on the transmissive cover.
(14) In (12) above, the flexible substrate is configured so as to be divided into at least a left section and a right section, and a lead wire for supplying electric power to the LEDs is disposed between the left and right sections of the flexible substrate that is divided in two,
   a shaft body, positioned in the vertical direction, is provided between the left and right sections of the flexible substrate, and
   a heat-insulating, light-blocking sponge is disposed on the housing-side of the shaft body, and a heat-insulating, a light-blocking sponge is disposed between the housing and each of the left and right sections of the flexible substrate with the lead wire positioned therebetween.
(15) In (1) above, an opening section which opens an eye region to the outside is formed in the housing, and an eye cup which blocks external light is provided to at least a portion of the opening section.
(16) In (13) above, an electrode attachment hole is formed in the transmissive cover, the electrode is attached in a non-removable manner in the electrode attachment hole with an electrode surface exposed to an inner surface-side, and a biasing member which biases the electrode toward the face is disposed between the electrode and the flexible substrate.

### BRIEF DESCRIPTION OF DRAWINGS

FIGS. 1(a) and (b) are, respectively, a front view and a rear view of the same mask-type cosmetic device.

FIG. 2 is a perspective view of the mask-type cosmetic device.

FIG. 3(a)-(d) are explanatory diagrams explaining folding patterns of the mask-type cosmetic device.

FIG. 4 is a front view of the mask-type cosmetic device with a fixing belt in an attached state.

FIG. 5 is a rear view of the mask-type cosmetic device with a fixing belt in an attached state.

FIG. 6(a)-(d) are exploded views of the mask-type cosmetic device.

FIG. 7 is an explanatory diagram explaining segments of a flexible substrate.

FIG. 8 is an upper-side view of the mask-type cosmetic device seen from above.

FIGS. 9(a) and (b) are explanatory diagrams of a folding section of a housing.

FIG. 10(a)-(c) are explanatory diagrams explaining a folding section and a planar part.

FIG. 11 (a)-(f) are explanatory diagrams explaining each part of the mask-type cosmetic device.

FIG. 12 is an exploded perspective view of the mask-type cosmetic device.

FIG. 13 is an exploded perspective view of the mask-type cosmetic device.

FIG. 14(a)-(f) are explanatory diagrams explaining folding section structures.

FIGS. 15(a) and (b) are explanatory diagrams explaining folding section structures.

FIGS. 16(a) and (b) are perspective views of a mask-type cosmetic device relating to a first modification of the present embodiment, with 16(a) showing a case in which a controller is worn, and 16(b) showing a case in which the controller is removed.

FIG. 17 is an explanatory diagram showing a nose ventilation section and a mouth ventilation section of the mask-type cosmetic device.

FIG. 18 is an explanatory diagram of a relevant section of a rear surface of the mask-type cosmetic device.

FIG. 19 is an explanatory diagram of a storage section of the mask-type cosmetic device.

FIG. 20 is an explanatory diagram of a storage section of the mask-type cosmetic device.

FIG. 21 is an explanatory diagram of a fixing belt of the mask-type cosmetic device.

FIGS. 22(a) and (b) are rear surface-side views of the mask-type cosmetic device, with 22(a) being a rear surface view and 22(b) being an enlarged view of a relevant section.

FIG. 23 is an explanatory diagram of an eye cup of the mask-type cosmetic device.

FIG. 24 is a B-B cross-sectional view of FIG. 22.

FIG. 25 is an explanatory diagram relating to LED light-emission in the mask-type cosmetic device.

FIG. 26 is an explanatory diagram relating to LED light-emission in the mask-type cosmetic device.

FIG. 27 is an explanatory diagram relating to an LED arrangement in a mask-type cosmetic device relating to a second modification of the present embodiment.

### DESCRIPTION OF EMBODIMENTS

Embodiments are explained in detail below with reference to the drawings. However, unnecessarily detailed explanations may be omitted. For example, detailed explanations of matters that are already known or explanations which would duplicate those for a configuration that is substantially the same may be omitted.

Note that the attached drawings and explanations thereof below are provided to enable a person of ordinary skill in the art to adequately understand the feature according to the present disclosure and are not intended to be used to limit the subject matter set forth in the claims.

A mask-type cosmetic device 1 is provided with a function of applying electrical stimulation to a face, and the electrical stimulation can be switched on and off. In a state in which the electrical stimulation is on, the output level of the electrical stimulation can be switched from stages 1 through 3.

The mask-type cosmetic device 1 is provided with green, red, and yellow as modes for irradiating light onto the face. Further, the mode for irradiating light can be changed in accordance with a part of the face, and parts that can be selected as a part of a face are the forehead, the area around the eyes, and the cheeks.

Further, skin temperature can be raised due to heat emission when electrical stimulation is applied to the face or due to light emission when light is irradiated.

A controller 27 (described later) of the mask-type cosmetic device 1 is an external component of the mask-type cosmetic device 1, is integrated with a housing 9 formed from a flexible material, and is provided with an indicator that displays the mode.

As a stimulation section 2 that applies electrical stimulation to the face or irradiates light onto the face, it is possible to use a halogen, an IPL, an organic EL, a laser, an interference wave, an ultrasonic wave, a sonic wave, vibration, heat, plasma, etc., without being limited thereto.

See FIG. 1(a), (b), and FIG. 3. FIG. 1(a) shows a view seen from the front surface of the mask-type cosmetic device 1 of the present example, and FIG. 1(b) shows a view seen from the rear surface of the mask-type cosmetic device 1 of the present example. FIG. 3 is a perspective view of the mask-type cosmetic device 1.

As shown in FIG. 1(b), the mask-type cosmetic device 1 includes: a plurality of electrodes 3 (capable of switching between three stages of Electric Muscle Stimulation (EMS), etc.; hereinafter referred to EMS electrodes) and a plurality of LEDs 5 (Light-Emitting Diode).

One LED 5 may include four chips (infrared in the range of 790-900 nm and preferably 850 nm; red in the range of 610-650 nm and preferably 630 nm; green in the range of 485-525 nm and preferably 505 nm; and yellow in the range of 570-610 nm and preferably 590 nm). In a mode for irradiating light onto the face, when, for example, the target parts of the face are assumed to be the forehead and the cheeks, the three above modes (green, red, and yellow) may be combined to be nine in total.

In addition, it is possible to perform a treatment with only electrical stimulation applied to the face, with only light irradiation being performed, and with both electrical stimulation and light irradiation being performed. Switching between the foregoing is performed by the controller 27.

For example, in an area E1, the forehead is treated and in an area E2, the cheeks are treated. It is also possible to switch between a treatment of the forehead only, a treatment of the area around the eyes only, a treatment of the cheeks only, and a treatment of all of the forehead, the area around the eyes, and the cheeks. It is also possible to perform a treatment by selecting any one or more parts of the face. When light irradiation is to be performed, it is possible to select a mode for each part that is to be irradiated. It is also possible to select the same mode for all parts. The control of mode switching is performed by the controller 27.

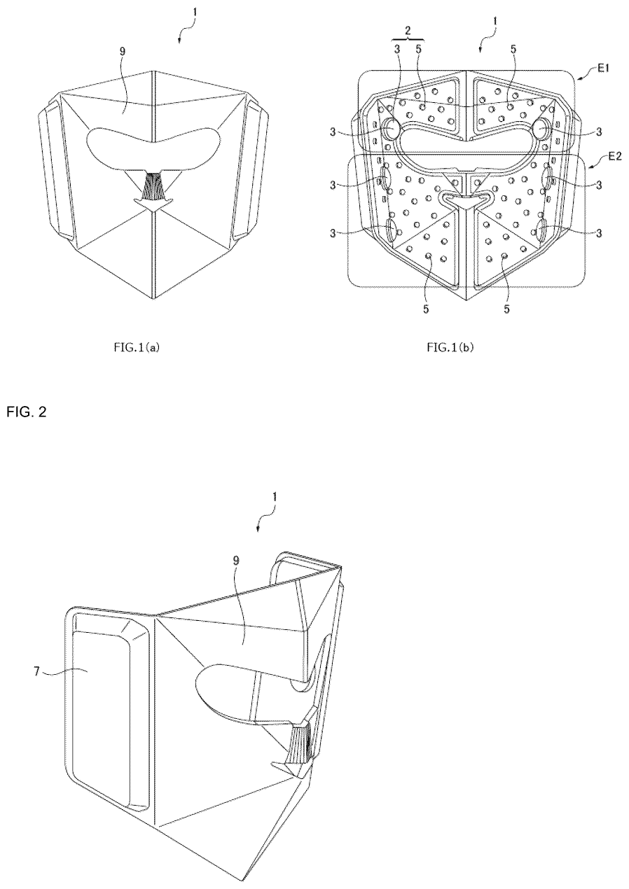

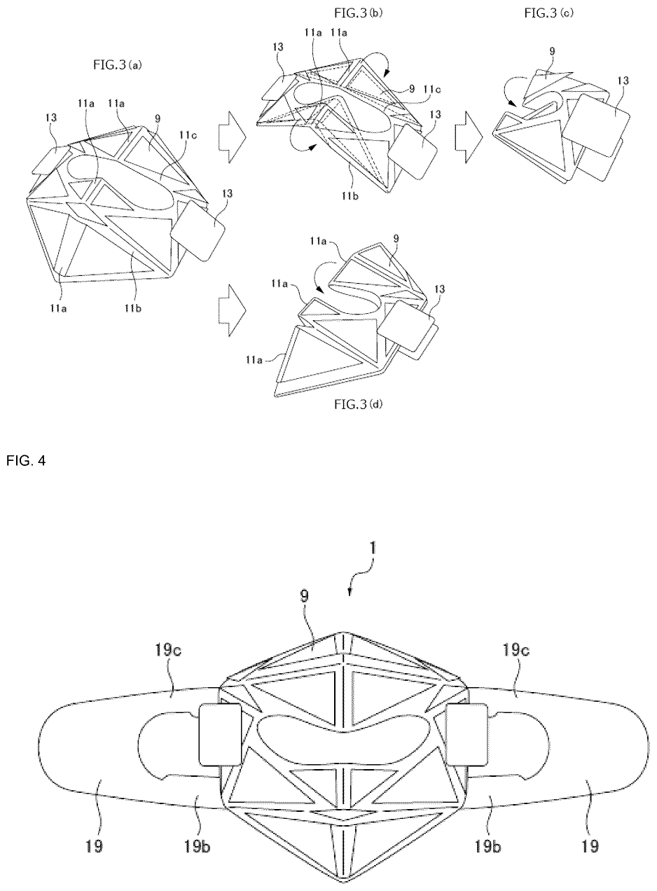

As shown in FIG. 2, in the mask-type cosmetic device 1, the housing 9 that covers an exterior section (flexible substrate 15, transmissive cover 17, which are described later, and face, etc.) and a storage section 7 (having the controller 27 built-in thereto) are provided in an integrated manner. A storage case may, for example, be provided as the storage section 7 but the storage section 7 is not limited thereto. The housing 9 of the mask-type cosmetic device 1 may be provided with a hollow space and may be provided with a slit or a pocket.

Alternatively, the controller 27 may be attached by providing a detachable part to a surface of the housing 9 of the mask-type cosmetic device 1. That is, there may be a configuration in which the storage section 7 is a separate body from the housing 9 and is detachable.

See FIGS. 3(a), (b), (c), and (d). FIG. 3 is diagram showing folding patterns of the mask-type cosmetic device 1.

As a first pattern, as shown in FIGS. 3(a), (b), and (c), a folding section 11a, a folding section 11b, and a folding section 11c are simultaneously folded to an inner surface-side (face-side) of the housing 9, and fixed by a pair of surface fasteners 13 provided to side sections (ear sections) of the housing 9. The mask-type cosmetic device 1 is configured so that, at that time, respective inner angles of two planes serving as references for the folding section 11a, the folding section 11b, and the folding section 11c are from 0 degrees to no more than 90 degrees. The angle of each inner angle is preferably from 0 degrees to no more than 45 degrees. Each angle is more preferably from 0 degrees to no more than 10 degrees.

Further, the flexible substrate 15 provided to an inner surface-side of the housing 9, is folded at a location between a flexible substrate 15a and a flexible substrate 15b corresponding to the folding section 11a. The flexible substrate 15 is divided into a plurality of sections (at least two divided sections) and has a flexible lead wire 24 (cable) which connects the plurality of divided flexible substrate 15 sections.

Furthermore, a transmissive cover 17 which is provided to an inner surface-side of the flexible substrate 15, is formed from silicon rubber, and transmits light from the LED 5 is also folded.

In such a manner, after the mask-type cosmetic device 1 is folded, the mask-type cosmetic device 1 is fixed and maintained in a folded state by the surface fasteners 13.

As a second pattern, as shown in FIGS. 3(a) and (d), only the folding section 11a is folded to the inner surface-side of the housing 9. The mask-type cosmetic device 1 is configured so that, at that time, respective inner angles of two planes serving as a reference for the folding section 11a are from 0 degrees to no more than 45 degrees. Meanwhile, the housing 9 has a flexible lead wire 24 which is flexible, can be folded, and connects the flexible substrate 15a and the flexible substrate 15b. In addition, the housing 9 is folded at a section between the flexible substrate 15a and the flexible substrate 15b through which the flexible lead wire 24 passes. Furthermore, the transmissive cover 17 formed from silicon rubber is also folded.

In such a manner, after the mask-type cosmetic device 1 is folded, the mask-type cosmetic device 1 is fixed and maintained in a folded state by the surface fasteners 13.

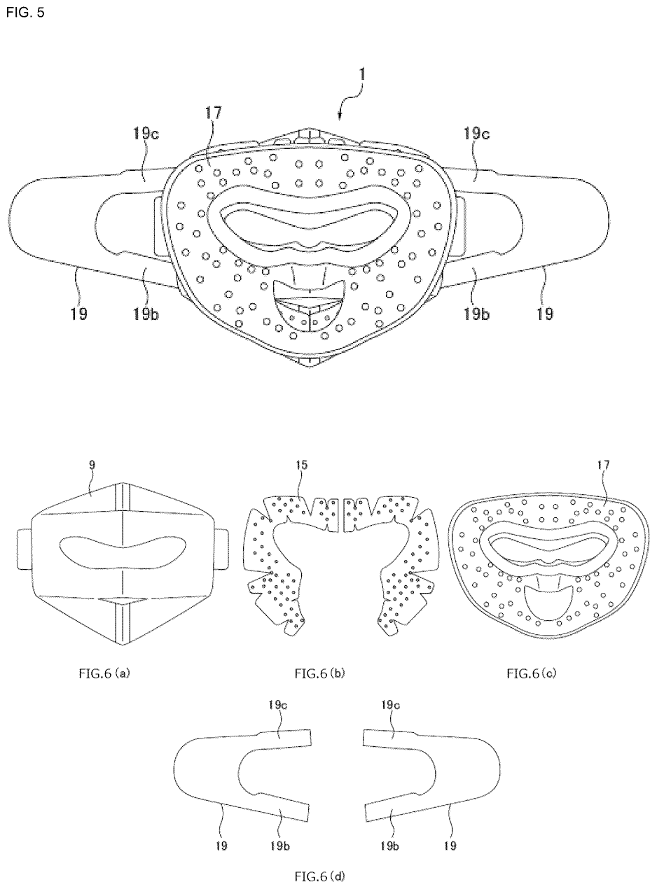

See FIGS. 4 and 5. FIG. 4 is a front view of the mask-type cosmetic device 1 provided with left and right fixing belts 19. FIG. 5 is a rear view of the mask-type cosmetic device 1 provided with left and right fixing belts 19.

Each of the left and right fixing belts 19 is a fixing means provided to the housing 9 for fixing the mask-type cosmetic device 1 to the face and is a band-like member formed such that a dimension in the height direction increases toward the housing 9. Each of the left and right fixing belts 19 has a rectangular opening section at an ear section location when worn on the face. Further, a lower section 19b of each of the left and right fixing belts 19 may have a dimension in the height direction of an extent enabling the chin to be pulled up and held when worn on the face.

Further, the left and right fixing belts 19 may be inserted into the housing 9 and fixed by means of magnets, etc., and may be configured integrated with the housing. Flexible bands, etc., are used as the left and right fixing belts 19. Further, it is also possible to use a holding band, a magnet, and furthermore, a fastener, as a component that joins the left and right fixing belts 19 to one another. Further, another method may be used such as using Velcro® or tying the fixing belts to one another.

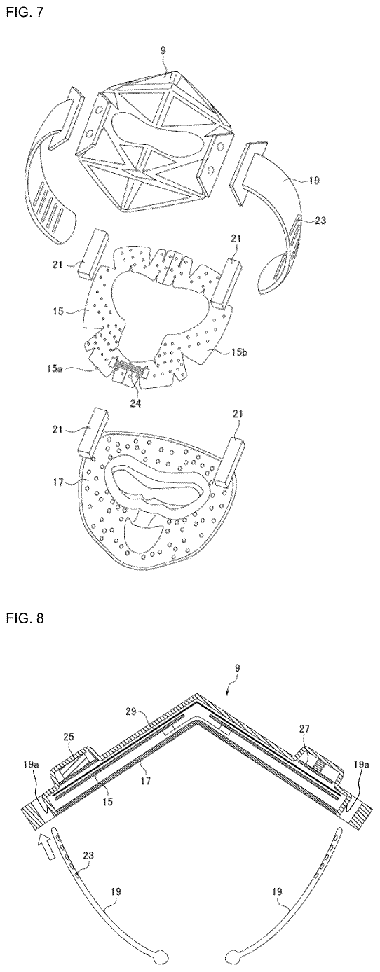

FIGS. 6(a), (b), (c), and (d) show the main constituent components of the mask-type cosmetic device 1. FIG. 6(a) shows the housing 9. FIG. 6(b) shows the flexible substrate 15. In the present example, for example, the flexible substrate 15 is divided into two sections. The details are described later. FIG. 6(c) shows the transmissive cover 17 which is formed from silicon rubber. FIG. 6(d) shows the left and right fixing belts 19. That is, the transmissive cover 17 contacts the face, the flexible substrate 15 is disposed so as to be on top of the transmissive cover 17, and finally is enveloped by the housing 9. Meanwhile, the left and right fixing belts 19 may be inserted into the housing 9 and fixed by means of magnets, etc., and may be rendered as integrated types. It is also possible to use a holding band, a magnet, and furthermore, a fastener, as a component that joins the left and right fixing belts 19 to one another. Further, another method may be used such as using Velcro® or tying the fixing belts to one another. Components such as a battery 25, the controller 27, and a coil spring 39, etc., are described later. Note that the battery 25 may be included in an externally provided controller 27 rather than in the housing 9.

See FIG. 7. In the present example, the flexible substrate 15 is divided into two sections. The two divided sections, namely, the flexible substrate 15a and the flexible substrate 15b are provided with an ABS resin fixing plate 21 interposed between each flexible substrate 15a, 15b, and the housing 9. Furthermore, the transmissive cover 17 is provided with ABS resin fixing plates 21 interposed between the transmissive cover 17 and each flexible substrate 15a, 15b.

By dividing the flexible substrate 15 into the flexible substrate 15a and the flexible substrate 15b, it is easy to fold the flexible substrate 15 together with the folding section 11a of the housing 9. Further, the flexible substrate 15a and the flexible substrate 15b are connected by the lead wire 24.

See FIG. 8. The left and right fixing belts 19 of the mask-type cosmetic device 1 are respectively inserted into left and right fixing belt insertion holes 19a and fixed by magnets 23 but there is no limitation to such a configuration and the left and right fixing belts 19 may be configured integrally with the housing 9. It is also possible to use a holding band, a magnet, and furthermore, a fastener, etc., as a component that joins the fixing belts 19 to one another. Further, another method may be used such as using Velcro® or tying the fixing belts 19 to one another. Further, the battery 25 and the controller 27 are stored in the housing 9 of the mask-type cosmetic device 1, and therefore, the battery 25, the controller 27, the flexible substrate 15a, and the flexible substrate 15b are connected. Note that the flexible substrate 15a and the flexible substrate 15b are connected by the lead wire 24, and the housing 9 is provided so as to cover the flexible substrate 15a and the flexible substrate 15b. However, a heat-shielding sheet 29, etc., may be disposed therebetween.

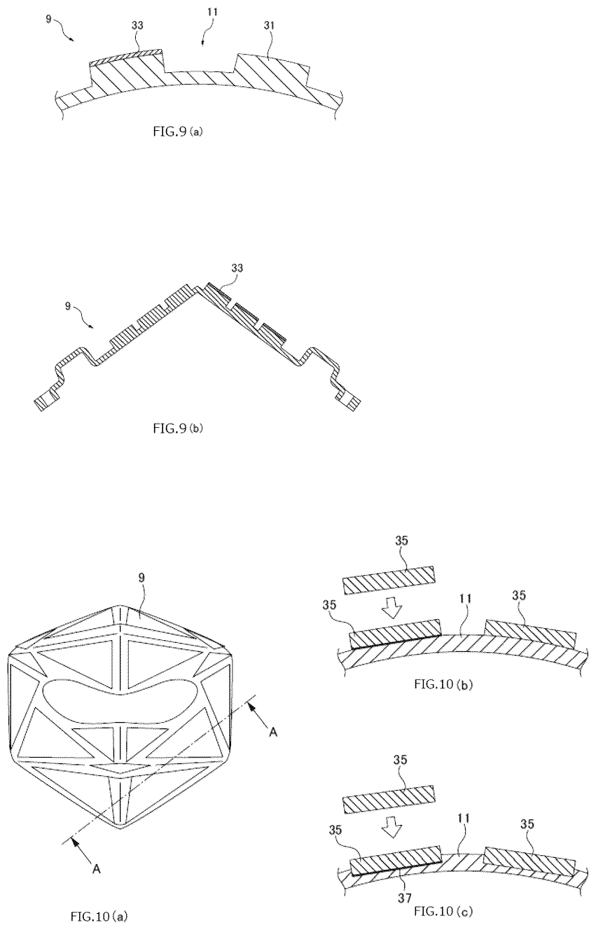

See FIGS. 9(a) and (b). The housing 9 formed of silicon rubber has folding sections 11 formed therein. However, the silicon rubber may have a plurality of protrusions 31 formed therein and recessed sections therebetween may serve as the folding sections 11. By configuring the housing 9 so that, on an inner surface thereof, the flexible substrates 15a, 15b are adjacent to one another with fine grooves, i.e., the recessed sections, therebetween, the fine grooves between the flexible substrates 15 serve as the folding sections 11.

Note that the protrusions 31 are a design surface, and therefore, printing 33 is applied to a surface thereof.

See FIGS. 10(a), (b), and (c). The cross-section A-A in FIG. 10(a) is shown divided into two patterns: FIG. 10(b) and FIG. 10(c). As shown in FIG. 10(b), an adjustment is made wherein by attaching, to the outermost side of the housing 9, planar parts 35 (plastic top) having a higher hardness than the housing 9, folding is made easier. Due to the presence of the planar parts 35, folding locations can be easily identified and it is also possible to prevent the flexible substrate 15a and the flexible substrate 15b from breaking. An adhesive agent 37 is used to attach the planar parts 35. In another pattern, as shown in FIG. 10(c), in the case of the housing 9 being silicon rubber, etc., the planar parts 35 are attached, using the adhesive agent 37, at locations so as to be half-buried in the housing 9. The mask-type cosmetic device 1 maintains a three-dimensional shape due to the planar parts 35. Further, the planar parts 35 may be configured so as to have a plurality of sizes and so as to be bilaterally symmetrical.

Furthermore, rather than having the planar parts 35, the mask-type cosmetic device 1 may be configured so as to have a large number of surfaces in which asperities of different thicknesses are formed in the housing 9.

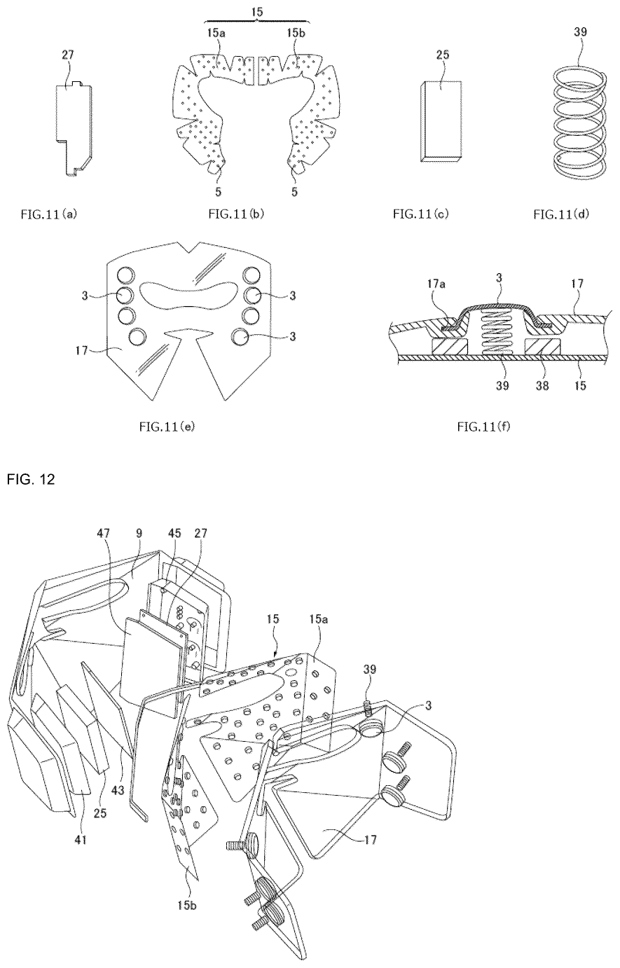

See FIGS. 11(a), (b), (c), (d), (e), and (f). FIG. 11(a) shows the controller 27. The controller 27 controls stimulation sections 2 provided with EMS electrodes 3, which are electrical stimulation sections that apply electrical stimulation to the face, and/or LEDs 5, which are light-emitting sections that irradiate light onto the face.

Further, the flexible substrate 15 is provided on an inner surface-side of the housing 9 and a plurality of LEDs constituting light-emitting sections are provided on the flexible substrate 15. Further, the transmissive cover 17 is provided on an inner surface-side of the flexible substrate 15 and the EMS electrodes 3 constituting some of the electrical stimulation sections are provided on the transmissive cover 17.

As shown in FIG. 11(b), the flexible substrate 15 is divided into two sections: the flexible substrate 15a and the flexible substrate 15b. The housing 9 is configured by having the flexible substrate 15 and furthermore, the transmissive cover 17 provided thereto with ABS resin fixing plates 21 interposed between the housing 9 and the flexible substrate 15, and between the flexible substrate 15 and the transmissive cover 17. By dividing the flexible substrate 15 into the flexible substrate 15a and the flexible substrate 15b, it is easy to fold the flexible substrate 15 together with the folding sections 11 of the housing 9.

FIG. 11(c) shows the battery 25. The battery 25 supplies electric power to the controller 27, the flexible substrate 15, the LEDs 5, and the EMS electrodes 3, etc.

FIG. 11(d) shows the coil spring 39 (biasing member). As shown in FIGS. 11(e) and (f), a plurality of EMS electrodes 3 are provided on the transmissive cover 17 and are electrically connected to the flexible substrate 15 via the coil spring 39. Further, an electrode attachment hole 17a is formed in the transmissive cover 17, the EMS electrode 3 is attached in a non-removable manner in the electrode attachment hole 17a with an electrode surface exposed to an inner surface-side, and the coil spring 39 which biases the EMS electrode 3 toward the face is disposed between the EMS electrode 3 and the flexible substrate 15.

Further, a sponge 38 is provided between the flexible substrate 15 and the transmissive cover 17 to which the EMS electrode 3 is attached so that the EMS electrode 3 and the flexible substrate 15 do not come into contact when the EMS electrode is pushed in by the face. Due to the sponge 38, even if the EMS electrode 3 is pushed in by the face, there is no contact with the flexible substrate 15, and heat from the flexible substrate 15 is unlikely to be transmitted to the EMS electrode 3.

Further, a cosmetic product (not shown in the drawings) is applied to a surface of the EMS electrode 3 or a conductive gel pad (not shown in the drawings) is adhered thereto. Due there, the EMS electrode 3 comes into contact with the face via the conductive gel pad, etc. In a case in which a conductive gel pad is adhered, in order to improve adhesion between the EMS electrode 3 and the conductive gel pad, a carbon sheet (not shown in the drawings) may be disposed on a surface of the EMS electrode 3. Note that a sheet mask impregnated with a cosmetic product may be adhered to the entire surface of the inner surface-side of the housing 9. In addition, a conductive gel pad may be adhered to the entire surface of the inner surface-side of the housing 9. Further, rather than applying a cosmetic product to a surface of the EMS electrode 3, a cosmetic product may be applied to the face.

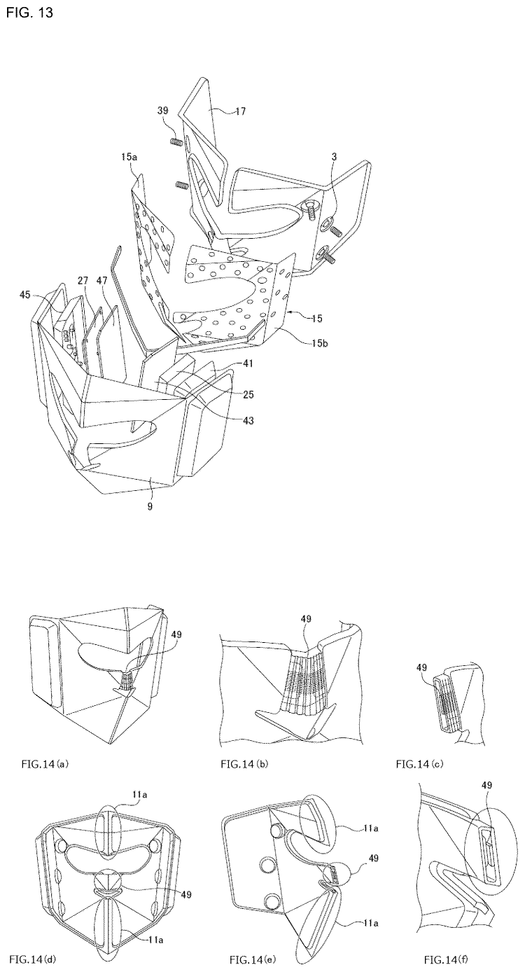

See FIGS. 12 and 13. The housing 9 of the mask-type cosmetic product 1 is provided at a location furthest from the face. The housing 9 includes a space for storing the battery surrounded by a case 41 and a case 43. Furthermore, there is provided a storage section 7 for storing the controller 27 surrounded a case 45 and a case 47.

In addition, the flexible substrate 15a and the flexible substrate 15b are disposed on an inner surface-side of the housing 9. The flexible substrate 15 is divided into two sections: the flexible substrate 15a and the flexible substrate 15b. In addition, the transmissive cover 17 is provided at a location in contact with the face. The mask-type cosmetic device 1 is configured by integrating the housing 9, the flexible substrate 15, and the transmissive cover 17.

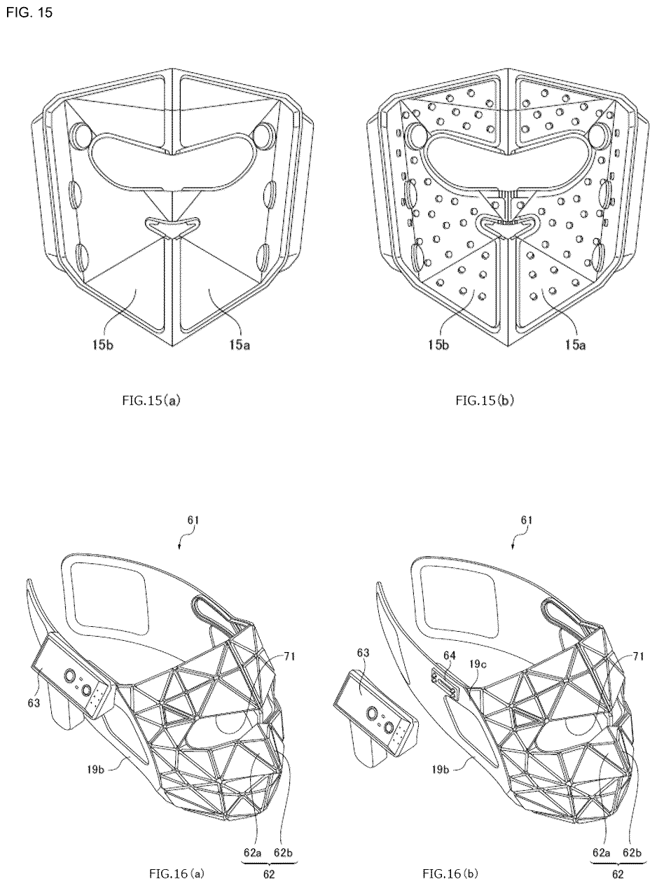

See FIGS. 14 and 15. In order to make it easy to fold the mask-type cosmetic device 1, it is desirable that there are as few layers as possible at a folding location. In order to achieve the foregoing, the lead wire 24 is disposed in a folding section 49 that contacts a nose section. However, it is also possible to set a configuration in which the folding section 49 folds easily by eliminating the housing 9 and configuring the mask-type cosmetic device 1 from the transmissive cover 17 and the lead wire 24.

Due to such a configuration, the mask-type cosmetic device 1 is folded flat when not used and is opened out to a three-dimensional shape when used. Further, the folding section 11 is disposed such that, when in use, the mask-type cosmetic device 1 is opened out to a three-dimensional shape by each surface adjacent to the folding section 11 being pulled centered on the folding section 11 of the housing 9. Further, the mask-type cosmetic device 1 is formed so that the size thereof when the housing 9 is folded flat is no more than 50% the size thereof when the housing 9 is opened out to a three-dimensional shape.

In such a manner, there is obtained a mask-type cosmetic device 1 which, when removed from the face, can be folded and stored even in a small space, and in which dust, etc., does not adhere to inner sections thereof.

### First Modification

Next, there follows a description of a first modification of the mask-type cosmetic device 1 described above.

See FIGS. 16, 17, 18, and 19.

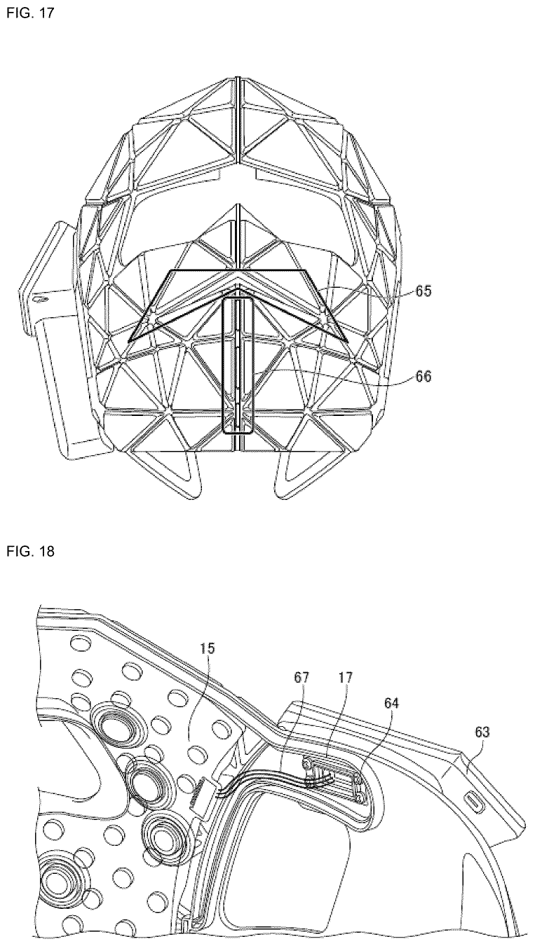

FIG. 16 shows perspective views of a mask-type cosmetic device according to a first modification of the present embodiment, with (a) showing a case in which a controller is worn, and (b) showing a case in which the controller is removed. FIG. 17 is an explanatory diagram showing a nose ventilation section and a mouth ventilation section of the mask-type cosmetic device. FIG. 18 is an explanatory diagram of a relevant section of a rear surface of the mask-type cosmetic device.

The differences between a mask-type cosmetic device 61 of the present modification compared to the present embodiment are described below.

See FIGS. 16, 17, and 18. A housing 62 of the mask-type cosmetic device 61 has a configuration in which a plurality of polygonal 62a regions including triangles are disposed adjacent to one another and fine grooves 62b are formed between each of the regions, and sections where the fine grooves 62b are formed are rendered thin so as to constitute the folding sections 11. Due thereto, the housing 62 can be formed as a single article without requiring any planar parts 35.

Note that in the housing 62 of the mask-type cosmetic device 61, the sections where the fine grooves 62b are formed are rendered thin. However, the housing 62 is not limited to such a case, and the folding sections 11 may also be configured by making the stiffness of the sections where the fine grooves 62b are formed lower than the stiffness of the polygonal 62a regions.

Further, the housing 62 is provided with a nose ventilation section 65 and a mouth ventilation section 66. The nose ventilation section 65 and the mouth ventilation section 66 facilitate breathing when the mask-type cosmetic device 1 is worn.

A detachable storage section 63 which is separate from the housing 62 is provided to an upper section 19c of either one of the left and right fixing belts 19. The storage section 63 is formed as a case which can store the controller 27 and the battery 25.

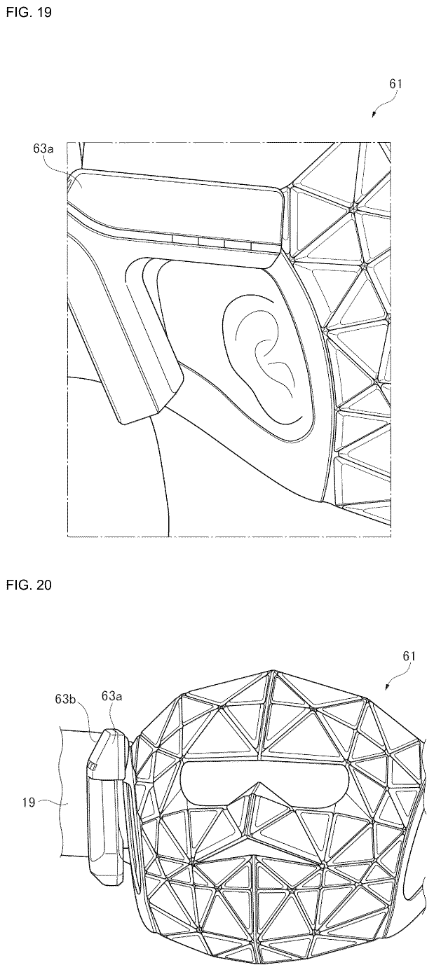

Note that the shape of the storage section 63 is not limited to an L-shape and may be a V-shape as shown in FIG. 19. The V-shaped storage section 63a is attached so as to conform with a human head, and therefore, can be worn easily. Further, as shown in FIG. 20, an operation surface 63b of the storage section 63a is attached so as to be angled in the height direction. Due thereto, when a button (not shown in the drawings) on the operation surface 63b is pressed while the mask-type cosmetic device 61 is being worn, a force for pressing the button of the operation surface 63b is conveyed diagonally downwardly rather than horizontally, and therefore, a load is unlikely to be applied to the head. A detachable section 64 for detachably attaching the storage section 63 is provided to the upper section 19c of either one of the left and right fixing belts 19. By providing the storage section 63 to the detachable section 64, flexibility of the upper section 19c of the fixing belt 19 to which the detachable section 64 is provided is lost. However, flexibility of a lower section 19b of the fixing belt 19 can be maintained. On an inner surface-side (face side) of the upper section 19c of the fixing belt 19 to which the detachable section 64 is provided, the transmissive cover 17 is provided extending as far as the detachable part 64, and one end of a lead wire 67 is connected to the detachable part 64. Further, the other end of the lead wire 67 is joined to the flexible substrate 15. Due thereto, the controller 27, the battery 25, and the flexible substrate 15 can be connected.

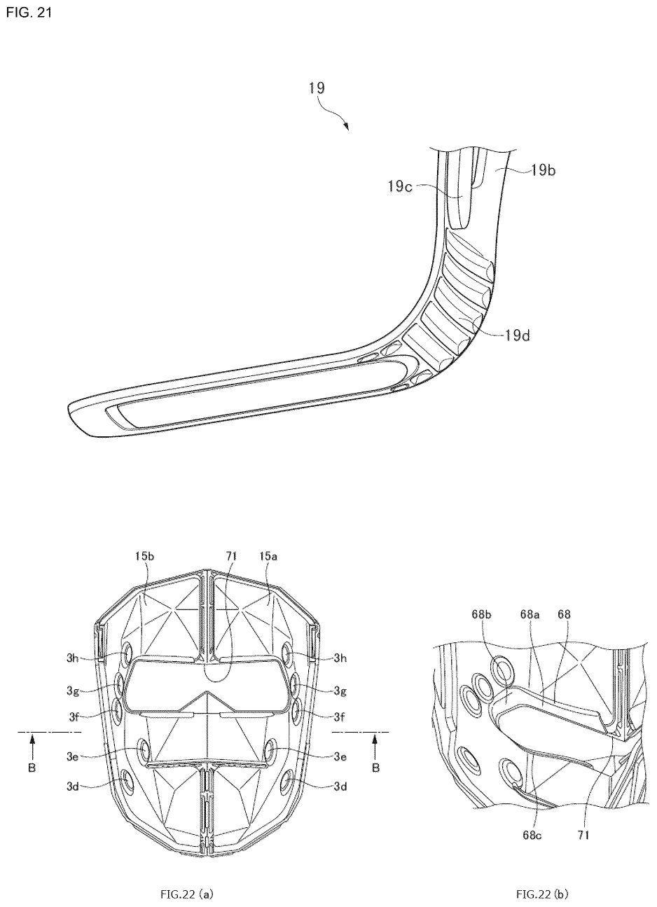

Further, as shown in FIG. 21, there is a ridged section 19d in the left and right fixing belts 19, and therefore, the left and right fixing belts 19 can be easily bent in a wrapping direction.

See FIG. 22(a). The EMS electrode 3 is provided with a first electrode 3f, a second electrode 3g, a third electrode 3h, a fourth electrode 3d, and a fifth electrode 3e. The first electrode 3f, the second electrode 3g, and the third electrode 3h are attached to the transmissive cover 17 so as to apply electrical stimulation to the cheeks. The fourth electrode 3d and the fifth electrode 3e are attached to the transmissive cover 17 so as to apply electrical stimulation to the area around the eyes.

See FIG. 22(b). An opening section 71 which opens an eye region to the outside is formed in the housing 62, and an eye cup 68 which blocks external light is provided to at least a portion of the opening section 71.

The eye cup 68 is convex toward the face, and is formed from an eye section upper section 68a, side section 68b (ear side), and lower section 68c. The eye cup 68 is not provided to the nose section and does not cause any obstruction when the housing 62 is folded in a left-right manner.

Note that, as shown in FIG. 23, the eye cup 68 may be provided so that the left and right sections thereof are continuous with the nose section. In such a case, the eye cup 68 has connecting sections 68d which connect, respectively, the left and right upper sections 68a and the left and right lower sections 68c, and light-blocking properties are improved. Further, the connecting sections 68d are shaped so as to be foldable at the center of the left-right direction, and thus, do not obstruct folding of the mask-type cosmetic device 61.

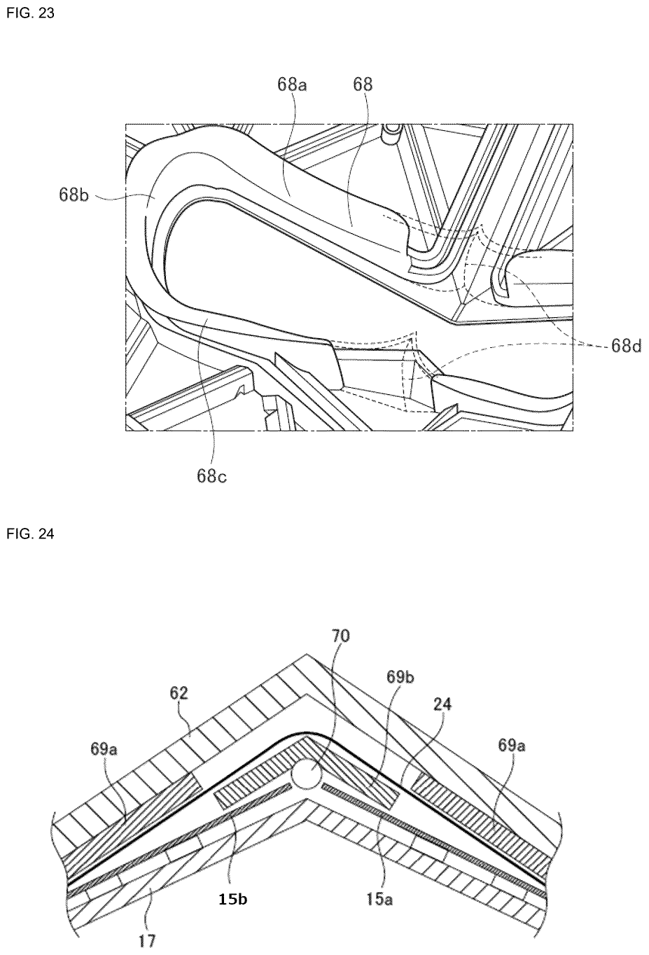

See FIG. 24. Between the housing 62 and the flexible substrate 15, there are provided: three heat-insulating, light-blocking sponges 69, a shaft body 70, and a lead wire 24.

Among the heat-insulating, light-blocking sponges 69, a pair of heat-insulating, light-blocking sponges 69a are provided adjacent to the inner surface of the housing 62 at positions separated from one another in the left-right direction from a central position where the housing 62 is folded in a left-right manner. Further, at a central position where the housing 62 is folded in a left-right manner, a heat-insulating, light-blocking sponge 69b is provided at a position separated from the inner surface of the housing 62, and thus, there are three heat-insulating, light-blocking sponges 69 in total.

The shaft body 70 is provided, at a central position where the housing 62 is folded in a left-right manner, so as to extend vertically. The shaft body 70 is provided adjacent to the heat-insulating, light-blocking sponge 69b on an inner surface-side (face side) of the heat-insulating, light-blocking sponge 69b.

The lead wire 24 is provided so as to pass the inner surface-side of the heat-insulating, light-blocking sponges 69a and pass the housing 62-side of the heat-insulating, light-blocking sponge 69b.

Due thereto, when the housing 62 is folded, the extent to which the lead wire 24 is bent is mitigated by the heat-insulating, light-blocking sponges 69 and the shaft body 70, and there is a lower risk of the lead wire 24 breaking due to an excessive force being applied thereto.

Further, the heat-insulating, light-blocking sponges 69 undertake a role of preventing heat from being transmitted from the flexible substrate 15 to the housing 62-side, and a role of preventing light emitted by the LEDs 5 from leaking to the housing 62-side.

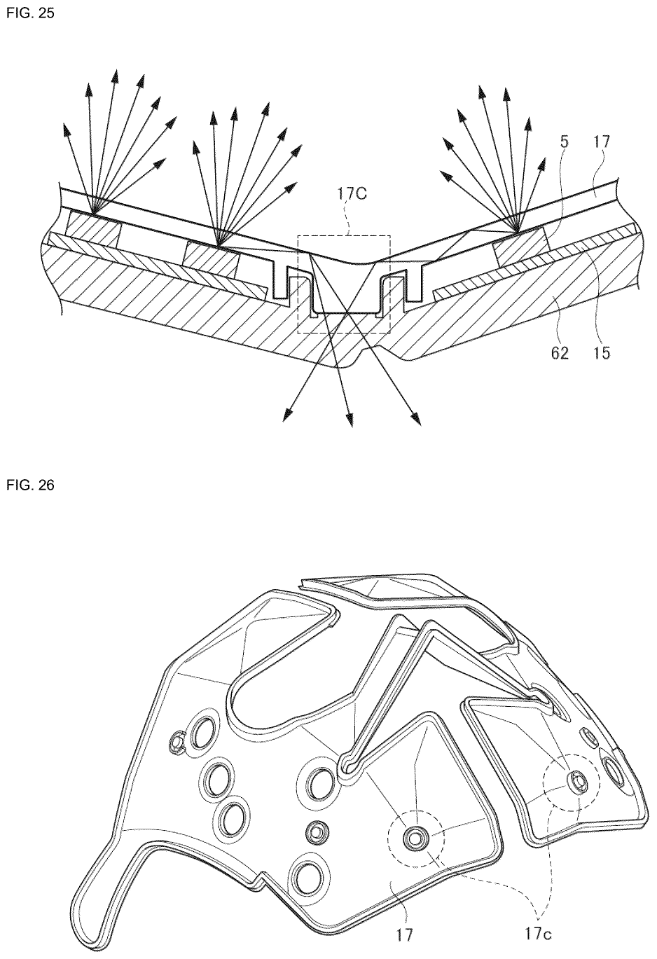

Note that the mask-type cosmetic device 61 may be configured so as to transmit light emitted by the LEDs 5 to the housing 62-side. Specifically, as shown in FIGS. 25 and 26, when the LEDs 5 emit light, the light passes through the transmissive cover 17 and advances from a protrusion 17c on the transmissive cover 17 toward the housing 62, and therefore, it is possible to recognize from the outside that light is emitted by the LEDs 5. Further, when the color of the light emitted by the LEDs 5 is close to the color of the housing 62 (for example, in the case of a red LED 5 and a red housing 62), the light of the LED 5 is easily transmitted.

According to such a configuration, the mask-type cosmetic device 61, like the present embodiment, can be folded easily. Further, the outer appearance of the housing 62 is expressed as a tile shape and is also an excellent design surface.

In the present modification, too, the mask-type cosmetic device 61 can be folded easily and it is possible to protect the inner sections thereof from dust, etc.

### Second Modification

Next, there follows a description of a second modification of the mask-type cosmetic device 1 described above.

The differences compared to the first modification are described below.

In the second modification, the arrangement of the LEDs 5 is determined on the basis of a power supply voltage (V), a voltage Vf for causing the LEDs 5 to emit light, and the number of LEDs 5 used.

Specifically, suppose, for example, that the power supply voltage is 6.4 V and nine LEDs 5 are required for each part (forehead, cheeks, etc.) of the face. Further, suppose that the light emission voltage Vf of an LED 5 infrared chip is 1.45 V, and a consumption current (A) is 0.06 A.

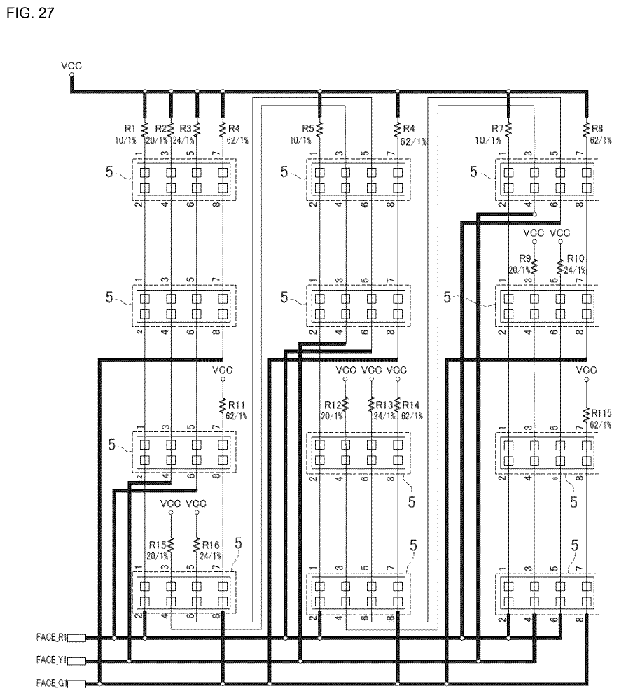

In such a case, as shown in FIG. 27, there is an arrangement having four LED 5 infrared chips connected in series (power supply voltage of 6.4 V>light emission voltage Vf of 1.45 V×four series-connected infrared chips) and three LED 5 infrared chips connected in parallel (four series-connected LEDs×three parallel-connected LEDs>number of LEDs 5 (nine)÷four series-connected LEDs 5).

The consumption current for such a case is determined by multiplying the consumption current by the number of LEDs 5 connected in parallel, and therefore, is the consumption current 0.06 A×three parallel-connected LEDs=0.18 A. Consumption current values in infrared chips at other parts of the face are derived by using the same calculation. In addition, the consumption current values for each part of the face are added together and the sum thereof is the total consumption current of the infrared chips.

Due thereto, a consumption power (W) of the infrared chips is determined by the total consumption current (A) of the infrared chips×the power supply voltage of 6.4 V.

Respective consumption powers of red chips, yellow chips, and green chips of the LEDs 5 are derived by using the same calculation, and the sum thereof is the total consumption power, and thus, the consumption power of the battery 25 is determined.

With respect to the red chips, for example, when the light emission voltage Vf of the red chip of the LED 5 is 2.00 V and the consumption current (A) is 0.03 A, there is an arrangement having three LED 5 red chips connected in series (power supply voltage of 6.4 V>light emission voltage Vf of 2.00 V×three series-connected red chips) and four LED 5 red chips connected in parallel (three series-connected LEDs×four parallel-connected LEDs>number of LEDs 5 (nine)÷three series-connected LEDs 5).

The consumption current for such a case is determined by multiplying the consumption current by the number of LEDs 5 connected in parallel, and therefore, is the consumption current 0.03 A×four parallel-connected LEDs=0.12 A.

Consumption current values in red chips at other parts of the face are derived by using the same calculation. In addition, the consumption current values for each part of the face are added together and the sum thereof is the total consumption current of the red chips.

Due thereto, the consumption power (W) of the red chips is determined by the total consumption current (A) of the red chips×the power supply voltage of 6.4 V.

With respect to the yellow chips, for example, when the light emission voltage Vf of the yellow chip of the LED 5 is 1.90 V and the consumption current (A) is 0.025 A, there is an arrangement having three LED 5 yellow chips connected in series (power supply voltage of 6.4 V>light emission voltage Vf of 1.90 V×three series-connected chips) and four LED 5 yellow chips connected in parallel (three series-connected LEDs×four parallel-connected LEDs>number of LEDs 5 (nine)÷three series-connected LEDs 5).

The consumption current for such a case is determined by multiplying the consumption current by the number of LEDs 5 connected in parallel, and therefore, is the consumption current 0.025 A×four parallel-connected LEDs=0.10 A.

Consumption current values in yellow chips at other parts of the face are derived by using the same calculation. In addition, the consumption current values for each part of the face are added together and the sum thereof is the total consumption current of the yellow chips.

Due thereto, the consumption power (W) of the yellow chips is determined by the total consumption current (A) of the yellow chips×the power supply voltage of 6.4 V.

With respect to the green chips, for example, when the light emission voltage Vf of the green chip of the LED 5 is 3.10 V and the consumption current (A) is 0.025 A, there is an arrangement having two LED 5 green chips connected in series (power supply voltage of 6.4 V>light emission voltage Vf of 3.10 V×two series-connected chips) and six LED 5 green chips connected in parallel (two series-connected LEDs×six parallel-connected LEDs>number of LEDs 5 (nine)÷two series-connected LEDs 5).

The consumption current for such a case is determined by multiplying the consumption current by the number of LEDs 5 connected in parallel, and therefore, is the consumption current 0.025 A×six parallel-connected LEDs=0.15 A.

Consumption current values in green chips at other parts of the face are derived by using the same calculation. In addition, the consumption current values for each part of the face are added together and the sum thereof is the total consumption current of the green chips.

Due thereto, the consumption power (W) of the green chips is determined by the total consumption current (A) of the green chips×the power supply voltage of 6.4 V.

Note that in the LED 5 arrangement, the power supply voltage that can realize the most efficient arrangement is, for example, 6.4 V, but may be a different value.

The light emission voltage Vf that is considered to be efficient for each of the LED 5 chips is in the range of 0.04-2.40 V for the infrared chips, is in the range of 0.10-3.40 V for the red chips, is in the range of 0.10-3.40 V for the yellow chips, and is in the range of 0.02-4.40 V for the green chips.

Note that when the power supply voltage is 6.4 V, for example, the light emission voltage Vf of each of the LED 5 chips is in the range of 1.00-1.59 V for the infrared chips, is in the range of 1.00-2.10 V for the red chips, is in the range of 1.00-2.10 V for the yellow chips, and is in the range of 1.00-3.19 for the green chips.

According to a configuration such as that described above, the LEDs 5 can be arranged efficiently (with efficient consumption power). Specifically, the LEDs 5 should be arranged in a number having a common multiplier (for example, 12, 24, 36, etc.) for four infrared chips that can be arranged in series, three red chips and yellow chips that can be arranged in series, and two green chips that can be arranged in series.

Further, one LED 5 is provided with four chips (infrared chip, red chip, yellow chip, and green chip).

Due thereto, it is possible to exert a higher anti-aging on human skin than a general LED that has three chips (red chip, yellow chip, and green chip).

Further, by fixing the power supply voltage (for example, 6.4 V) rather than preparing different voltages for each chip, the controller 27 can be made smaller. Note that the power supply voltage does not have to be fixed.

Furthermore, the second modification can be applied not only in a case in which the mask-type cosmetic device 1, 61 is to be folded but also in a case in which the mask-type cosmetic device 1, 61 is configured so as not to be folded.

The main points described above shall now be summarized.
(1) One aspect is a mask-type cosmetic device 1, 61 that can be worn on a face, wherein: the mask-type cosmetic device 1, 61 includes a housing 9, 62 which covers an exterior section, stimulation sections 2 including electrical stimulation sections which apply electrical stimulation to the face and/or light-emitting sections which irradiate light onto the face, a controller 27 which controls the stimulation sections 2, and a fixing means (left and right fixing belts 19) which is provided to the housing 9, 62 and fixes the mask-type cosmetic device 1, 61 to the face; and a section that is worn on the face and includes the housing 9, 62 is configured so as to be foldable.
(2) In (1) above, the housing 9, 62 has provided thereto a storage section 7 for storing the controller 27 integrally with the housing 9, 62 or separately therefrom in a detachable manner.
(3) In (1) above, the fixing means is configured as left and right fixing belts 19 which are integral with left and right sections of the housing 9, 62 or are provided in a detachable manner thereto, and one of the fixing belts 19 has provided thereto a storage section 7 for storing the controller integrally with the fixing belt 19 or separately therefrom in a detachable manner.
(4) In (1) above, the housing 9, 62 has foldable folding sections 11.
(5) In (4) above, an inner angle of at least some of the folding sections 11 is from 0 degrees to no more than 45 degrees.
(6) In (4) above, the housing 9, 62 is formed from a flexible material and has a configuration in which a plurality of substrates (flexible substrate 15) are disposed, on an inner surface of the housing 9, 62, adjacent to one another and separated by fine grooves, and the fine grooves between the plurality of substrates constitute the folding sections 11.
(7) In (4) above, the housing 62 has a configuration in which a plurality of polygonal regions are provided adjacent to one another and fine grooves are formed between each of the regions, and sections where the fine grooves are formed are rendered thin so as to constitute the folding sections 11.
(8) In (4) above, the housing has a configuration in which a plurality of polygonal regions are provided adjacent to one another and fine grooves are formed between each of the regions, and
   sections where the fine grooves are formed have a stiffness that is lower than the stiffness of the regions so as to constitute the folding sections.
(9) In (4) above, the mask-type cosmetic device 1, 61 is folded flat when not used and is opened out to a three-dimensional shape when used.
(10) In (9) above, the folding sections 11 are disposed such that, when used, the mask-type cosmetic device 1, 61 is opened out to a three-dimensional shape by pulling out the housing.
(11) In (9) above, the mask-type cosmetic device 1, 61 is configured such that size thereof when the housing 9, 62 is folded flat is no more than 50% the size thereof when the housing 9, 62 is opened out to a three-dimensional shape.
(12) In (1) above, a flexible substrate 15 is disposed on an inner surface-side of the housing 9, 62, and a plurality of LEDs 5 which constitute the light-emitting sections are disposed on the flexible substrate 15.
(13) In (12) above, a transmissive cover 17 is disposed on an inner surface-side of the flexible substrate 15, and EMS electrodes 3 constituting some of the electrical stimulation sections are disposed on the transmissive cover 17.
(4) In (12) above: the flexible substrate 15 is configured so as to be divided into at least a left section and a right section, and a lead wire 24 for supplying electric power to the LEDs 5 is disposed between the left and right sections of the flexible substrate 15 that is divided into two; a shaft body 70, positioned in the vertical direction, is provided between the left and right sections of the flexible substrate 15; and a heat-insulating, light-blocking sponge 69 is disposed on the housing 62-side of the shaft body 70, and a heat-insulating, light-blocking sponge 69 is disposed between the housing 62 and each of the left and right sections 15a, 15b of the flexible substrate with the lead wire 24 positioned therebetween.
(15) In (1) above, an opening section 71 which opens an eye region to the outside is formed in the housing 62, and an eye cup 68 which blocks external light is provided to at least a portion of the opening section 71.
(16) In (13) above, an electrode attachment hole 17a is formed in the transmissive cover 17, the EMS electrode 3 is attached in a non-removable manner in the electrode attachment hole 17a with an electrode surface exposed to an inner surface-side, and a coil spring 39 which biases the EMS electrode 3 toward the face is disposed between the EMS electrode 3 and the flexible substrate 15.

### INDUSTRIAL APPLICABILITY

As described above, the mask-type cosmetic device 1 according to the present disclosure can be folded easily and is also able to protect inner sections thereof from dust, etc., and therefore, can be utilized in various kinds of mask-type cosmetic devices for domestic use and industrial use.

### REFERENCE SIGNS LIST

1 Mask-type cosmetic device
2 Stimulation section
3 EMS electrode (electrical stimulation section)
5 LED (light-emitting section)
7 Storage section
9 Housing
11 Folding section
13 Surface fastener
15 Flexible substrate
17 Transmissive cover
19 Fixing belt
24 Lead wire
25 Battery
27 Controller
35 Planar part
38 Sponge
39 Coil spring
49 Folding section
61 Mask-type cosmetic device
62 Housing
63 Storage section
64 Detachable section
67 Lead wire
68 Eye cup
69 Heat-insulating, light-blocking sponge
70 Shaft body
71 Opening section

1. A mask-type cosmetic device that can be worn on a face, wherein:
the mask-type cosmetic device comprises
a housing which covers an exterior section,
stimulation sections comprising electrical stimulation sections which apply electrical stimulation to the face and/or light-emitting sections which irradiate light onto the face,
a controller which controls the stimulation sections, and
a fixing means which is provided to the housing and fixes the mask-type cosmetic device to the face; and
a section that is worn on the face and includes the housing is configured so as to be foldable.

2. The mask-type cosmetic device according to claim 1, wherein the housing has provided thereto a storage section for storing the controller integrally with the housing or separately therefrom in a detachable manner.

3. The mask-type cosmetic device according to claim 1, wherein:
the fixing means is configured as left and right fixing belts which are integral with left and right sections of the housing or are provided in a detachable manner thereto; and
one of the fixing belts has provided thereto a storage section for storing the controller integrally with the fixing belt or separately therefrom in a detachable manner.

4. The mask-type cosmetic device according to claim 1, wherein the housing has foldable folding sections.

5. The mask-type cosmetic device according to claim 4, wherein an inner angle of at least some of the folding sections is from 0 degrees to no more than 45 degrees.

6. The mask-type cosmetic device according to claim 4, wherein:
the housing is formed from a flexible material and has a configuration in which a plurality of substrates are disposed, on an inner surface of the housing, adjacent to one another and separated by fine grooves; and
the grooves between the plurality of substrates constitute the folding sections.

7. The mask-type cosmetic device according to claim 4, wherein:
the housing has a configuration in which a plurality of polygonal regions are provided adjacent to one another and fine grooves are formed between each of the regions; and
sections where the fine grooves are formed are rendered thin so as to constitute the folding sections.

8. The mask-type cosmetic device according to claim 4, wherein:
the housing has a configuration in which a plurality of polygonal regions are provided adjacent to one another and fine grooves are formed between each of the regions; and
the sections where the fine grooves are formed have a stiffness that is lower than the stiffness of the regions so as to constitute the folding sections.

9. The mask-type cosmetic device according to claim 4, wherein the mask-type cosmetic device is folded flat when not used and is opened out to a three-dimensional shape when used.

10. The mask-type cosmetic device according to claim 9, wherein the folding sections are disposed such that, when used, the mask-type cosmetic device is opened out to a three-dimensional shape by pulling out the housing.

11. The mask-type cosmetic device according to claim 9, wherein the mask-type cosmetic device is configured such that size thereof when the housing is folded flat is no more than 50% the size thereof when the housing is opened out to a three-dimensional shape.

12. The mask-type cosmetic device according to claim 1, wherein a flexible substrate is disposed on an inner surface-side of the housing, and a plurality of LEDs which constitute the light-emitting sections are disposed on the flexible substrate.

13. The mask-type cosmetic device according to claim 12, wherein a transmissive cover is disposed on an inner surface-side of the flexible substrate, and electrodes constituting some of the electrical stimulation sections are disposed on the transmissive cover.

14. The mask-type cosmetic device according to claim 12, wherein:
the flexible substrate is configured so as to be divided into at least a left section and a right section, and a lead wire for supplying electric power to the LEDs is disposed between the left and right sections of the flexible substrate that is divided in two;
a shaft body, positioned in the vertical direction, is disposed between the left and right sections of the flexible substrate; and
a heat-insulating, light-blocking sponge is disposed on the housing-side of the shaft body, and a heat-insulating, light-blocking sponge is disposed between the housing and each of the left and right sections of the flexible substrate with the lead wire positioned therebetween.

15. The mask-type cosmetic device according to claim 1, wherein an opening section which opens an eye region to the outside is formed in the housing, and an eye cup which blocks external light is provided to at least a portion of the opening section.

16. The mask-type cosmetic device according to claim 13, wherein: an electrode attachment hole is formed in the transmissive cover; the electrode is attached in a non-removable manner in the electrode attachment hole with an electrode surface exposed to an inner surface-side; and a biasing member which biases the electrode toward the face is disposed between the electrode and the flexible substrate.
